(19) United States
(12) Patent Application Publication
(10) Pub. No.: US 2007/0218155 A1
(43) Pub. Date: Sep. 20, 2007

(54) Methods and compositions for treating dyslipidaemia

(76) Inventor: Eric H. Kuhrts, Bodega, CA (US)

Correspondence Address: THORPE NORTH & WESTERN, LLP., 8180 SOUTH 700 EAST, SUITE 350, SANDY, UT, 84070, US

(21) Appl. No.: 11/701,963

(22) Filed: Feb. 1, 2007

Related U.S. Application Data
(62) Division of application No. 10/923,110, filed on Aug. 20, 2004, now abandoned.

(51) Int. Cl. A61K 36/00

(52) U.S. Cl. 424/778

(57) ABSTRACT

Disclosed are methods for lowering cholesterol and treating heart disease in an animal employing prenylchalcones or prenylflavonones. Such prenylchalcones or prenylflavonones may be derived from hops (humulus Lupulus L.), or produced synthetically. Representative prenylchalcones or prenylflavonones are: xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin. The preferred prenylchalcone is xanthohumol

### FIELD OF THE INVENTION

This invention relates to therapeutic compositions and methods for treating elevated cholesterol and heart disease.

### BACKGROUND OF THE INVENTION

Direct health care costs associated with cardiovascular disease exceed $100 billion per year in the United States alone and there are very few effective therapies available that treat more than one symptom or cause of coronary artery disease. Most of the drugs prescribed for heart disease treat one aspect of the disease such as elevated cholesterol, or blood pressure. Because insulin resistance and obesity are usually part of the same metabolic syndrome, therapeutic agents that attack the metabolic complications of cardiovascular disease, diabetes, and obesity would be of great value. The diabetic syndrome is usually accompanied by elevated levels of triglycerides and low levels of HDL cholesterol, a lipid profile that is considered to be one of dyslipidaemia, or a lipid profile associated with cardiovascular disease. Many patients with diagnosed coronary heart disease also have high cholesterol, in addition to low HDL and high triglycerides and fatty acids

Many large prospective clinical trials have demonstrated that reducing cholesterol levels in blood is effective treatment for the primary and secondary prevention of heart disease and other complications of atherosclerosis.

Triglycerides are the major storage form of energy and are synthesized primarily in three tissues; the small intestine, liver, and adipocytes. The major functions of the molecule in these tissues are: (a) dietary fat absorption, (b) lipoprotein packaging of the de novo synthesized fatty acids, and (c) fat storage in adipose tissue. Free fatty acids are converted from dietary fat through the digestion process by pancreatic lipase. Some fatty acids are produced endogenously from dietary carbohydrate that is not utilized for energy production. This takes place primarily in the liver. Fatty acid synthesis is greatly elevated by glucose and insulin.

Since triglycerides are the main form of storage of excess calories in fat, recent research has focused on the key enzyme responsible for the synthesis of triglycerides, acylCoA:diacylglycerol acyltransferase in cells, or DGAT.

DGAT is a microsomal enzyme that occurs throughout mammalian tissues, and is also responsible for catalyzing the final step in the monoacylglycerol pathway in the small intestine. Recently, the gene for DGAT has been identified and cloned, enabling molecular studies to be performed. Northern blot analysis of DGAT mRNA levels has revealed that this enzyme is expressed in all tissues examined, but exists in the highest levels in the liver, small intestine, and adipose tissue. DGAT expression was also detected in skeletal muscle and brain.

The DGAT gene has been inactivated in a special strain of mice, called DGAT knockout (Dgat-I-) mice. These mice have been used to study the function of DGAT, and the implications of its absence. DGAT knockout mice, or mice lacking the DGAT gene, were still healthy mice, but had less adipose tissue, and lower total fat pad weights and body triglyceride levels. When fed a high fat diet (21% fat by weight), these mice maintained the same weight as the group of non-knockout mice controls fed a regular chow diet consisting of 4% fat by weight. The other mice consuming the high fat diet, experienced a 40-50% weight gain. The weight difference was primarily related to about a 40% decrease in total carcass triglycerides in the DGAT knockout mice.

DGAT knockout mice also exhibit higher insulin sensitivity, indicating that triglyceride metabolism is tightly linked with glucose metabolism.

Another interesting feature that was discovered related to DGAT knockout mice is that DGAT deficiency improves glucose metabolism. In addition to having potential to effect weight loss and energy expenditure, deficiency in DGAT appeared to alter glucose metabolism in the knockout mice. These mice seem to have normal levels of plasma glucose and insulin, but when given a glucose load, had lower glucose and insulin levels than regular mice. This indicates that inhibition of DGAT enzyme could improve glucose metabolism. DGAT deficiency also lowered serum insulin levels in Agouti yellow mice. These mice are genetically obese and insulin resistant. Therefore, inhibition of DGAT could be an effective treatment strategy for diabetics by improving glucose metabolism.

To summarize, mice that are deficient in the DGAT enzyme are resistant to diet induced obesity and have increased insulin and leptin sensitivity. Research suggests that therapeutic inhibition of DGAT in-vivo may result in effective treatment for elevated triglycerides and the cardiovascular complications of diabetes. Therefore, an agent that inhibits DGAT, or a DGAT inhibitor would be of great utility for the treatment of diabetes, and heart disease. Inhibiting DGAT is also a fitting therapeutic strategy for a small molecule drug directed at the dyslipidaemia associated with the metabolic syndrome associated with diabetes and obesity.

Lipids are a group of fatty compounds that include phospholipids, triglycerides, and cholesterol (sterols). While cholesterol is a key constituent of cellular membranes, elevated cholesterol is a major risk factor for coronary artery disease or arteriosclerosis. Cholesterol and other fatty compounds (lipids) in the blood are insoluble, and require certain carriers that are capable of incorporating them into soluble complexes that can be transported to specific target sites. These soluble complexes are called lipoproteins. When lipids become pathogenic, through oxidation of cholesterol, or levels of cholesterol that are above a normal healthy level, the result is atherogenesis, or the development of heart disease and its various complications. Heart disease can also be the result of a disproportionate amount of various lipid fractions such as high density lipoprotein (HDL), low density lipoprotein (LDL), and triglycerides and other fatty acids. This disproportionate balance of lipids is known as dyslipidaemia.

One of the primary drug targets for treating hypercholesterolemia, or elevated blood cholesterol, has been the inhibition of HMG-CoA reductase (3-hydroxy-3-methylglutaryl CoA reductase), the enzyme in the liver that is responsible for the synthesis of mevalonic acid, and an intermediate in the biosynthesis of sterol (cholesterol). Currently, there are many approved HMG-CoA reductase inhibitor drugs (statins), such as lovastatin, simvastatin, pravastatin, fluvastatin, and atorvastatin. There are also a few HMG-CoA reductase inhibitors identified in natural sources such as plant extracts and red yeast rice (Monascus purpureus). The benefits of statin drugs in the primary and secondary prevention of heart disease have been shown in numerous, large prospective clinical trials.

Acyl-coenzyme A cholesterol acyl transferase (ACAT) is an enzyme that esterifies cholesterol. For unesterified “free” cholesterol to be packaged into ApoB-containing lipoproteins in the liver, it needs to be first esterified by ACAT. ACAT inhibition is believed to be antiatherogenic by accelerating cholesterol excretion by the liver, as well as by inhibiting cholesterol absorption in the intestines. ACAT inhibition also may prevent cholesteryl ester accumulation in macrophages in the arterial walls, which results in antiatherosclerosis effects. ACAT inhibition may have direct effects on the vascular system through impairment of conversion of free cholesterol to esterified cholesterol in endothelial macrophage by reducing foam cell formation.

Normally, ACAT inhibitors are thought to prevent accumulation of lipid in the arterial wall without significantly affecting plasma lipid levels. However, an agent that inhibits both ACAT and HMG CoA reductase, such as the compounds of this invention, will lower cholesterol and prevent accumulation of lipid in the arterial wall, in addition to lowering triglycerides and free fatty acids by inhibiting DGAT.

An excellent review article related to current therapies for treating dyslipidaemia is: Bays et al; Pharmacotherapy for Dyslipidaemia-Current Therapies and Future Agents; Expert Opin Pharmacother: 2003 4(11): 1901-1938, hereby incorporated by reference in its entirety.

### SUMMARY OF THE INVENTION

Accordingly, it is an object of the present invention to provide a novel method of treating elevated cholesterol, triglycerides, and cardiovascular disease in an animal by inhibiting HMG-CoA redutase (3-hydroxy-3-methylglutaryl CoA reductase), ACAT (acyl-coenzyme A cholesterol acyl transferase), and DGAT (acyl CoA:diacylglycerol acyltransferase), with an extract of hops, a chalcone or flavonoid derived from the hops plant (Humulus lupulus L.), an isolated prenylchalcone or prenylflavonones, or a synthetic prenylchalcone or prenylflavones. The prenylchalcone or prenylflavones may be derived from hops or extracted form other botanical sources that may contain the same compounds. The primary chalcones contained in hops that are effective for inhibiting HMG CoA reductase, ACAT, and DGAT are xanthohumol A and xanthohumol B, with xanthohumol A, the preferred chalcone. Other prenylchalcones or prenylflavonones, either alone or in combination may be used.

It is an additional object of the invention to provide formulations for treating the dyslipidaemia associated with diabetes.

### DETAILED DESCRIPTION OF THE INVENTION

Flavonoids are abundant throughout nature and exert a broad range of biological activities in plants and animals. There are now considered to be over 4,000 flavonoids existent in nature. Some of the biological activities of flavonoids include; anti-inflammatory, antiviral, antifungal, antibacterial, estrogenic, anti-oxidant, antiallergenic, anticarcinogenic, and antiproliferative medicinal properties.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, the more important ones being the chalcones or prenylflavonoids; xanthohumol, isoxanthohumol, desmethylxanthohumol, 8-prenylnaringenin, and 6-prenylnaringenin. Some of these prenylflavonoids exhibit potent estrogenic activity, such as 8- prenylnaringenin, and are considered to be phytoestrogens (Reproduction; 2002; 123, 235-242). Xanthohumol is the principle flavoniod contained in hops. Xanthohumol does not exhibit estrogenic activity (Journal of Endocrinology and Metabolism; 85; 12, 4912-4915).

Xanthohumol is a yellow-orange substance with a melting point of 172 degrees C. A typical ethanol extract of hops yields about 3 mg/g (3%) of xanthohumol out of a total flavonoid content of 3.46 mg/g. Dried hop contains about 0.2 to 1.0% by weight xanthohumol.

Typical Flavonoid Content of an ETOH Extract of Hops

| | | |
|---|---|---|
| Xanthohumol | 3 | mg/g |
| Desmethylxanthohumol | 0.34 | mg/g |
| Isoxanthohumol | 0.052 | mg/g |
| 6-prenylnaringenin | 0.061 | mg/g |
| 8-prenylnaringenin | 0.015 | mg/g |

Xanthohumol or the other prenylchalcones or prenylflavonones can be synthesized or isolated from hops through further purification, fractionation, or separation using methods that are known to those skilled in the art, or following the procedure of Tabata et. al.; Phytochemistry; 46, No. 4; pp. 683-687, 1997. Ethanol (EtOAc) or other solvents may be used to extract higher levels of the chalcones or flavones form hops. Supercritical carbon dioxide extractions, which do not use solvents, will tend to have much lower levels, or non-existent levels of the chalcones and flavonones. In fact these compounds are almost non-existent in standard CO2 extracts because the polyphenols (chalcones and flavonones) are virtually insolvent on carbon dioxide. Newer techniques of extraction using supercritical carbon dioxide may yield greater amounts of xanthohumol, or allow for the separation of xanthohumol and other flavonoids from other constituents of hops such as the alpha and beta acids, and essential oils and hard resins.

An excellent review of the flavonoids contained in the hop plant is contained in; Chemistry and Biology of Hops Flavonoids; Stevens, J. et. al.;. J. Am. Soc. Brew. Chem. 56 (4): 136-145, 1998, hereby incorporated by reference.

As used herein the term “chalcone” or “flavonone” refers to the following flavonoids; xanthohumol, xanthogalenol, desmethylxanthohumol(2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,s-diprenylnaringenin, 4′,6′-dimethoxy-2′,4-dihydroxychalcone,4′-O-methylxanthohumol, 6-geranylnaringenin, 8-geranylnaringenin.

As used herein, the term “HMG-CoA reductase inhibitor” refers to a substance that inhibits the activity of 3-hydroxy-3-methylglutaryl CoA reductase, a key enzyme in cholesterol synthesis. HMG-CoA reductase inhibition can be measured in-vitro in a suitable cell line such as HeG2 cells or rat liver microsomes.

As used herein, the term “ACAT inhibitor” refers to a substance that inhibits the activity of acyl-coenzyme A cholesterol acyl transferase, an enzyme that esterifies cholesterol.

As used herein, the term “DGAT inhibitor” refers to a substance that inhibits the activity of diacylglycerol acyltransferase, an enzyme involved in hypertriglyceridemia, or high levels of triglycerides and fatty acids, as well as fatty liver and obesity.

One method of determining if a compound is a DGAT inhibitor is the DGAT assay using rat liver microsomes. This assay was used by Tabata et. al. (Phytochemistry; 46; No. 4, 683-687, 1997) to screen xanthohumol for DGAT inhibition. Xanthohumol A and xanthohumol B inhibited DGAT activity with IC50 values of 50.3 and 194 pM respectively. The xanthohumols also showed preferential inhibition of triacylglycerol formation in intact Raji cells. Raji cells are intact cells and are used to assay for lipid formation. The Raji assay indicated that xanthohumol inhibited DGAT activity specifically in human cells.

### EXAMPLE 1

Human hepatopblastoma (HepG2) cells can be used to screen compounds for HMG CoA reductase inhibition activity. HepG2 cells can be obtained from the American Type Culture Collection (Rockville, Md.) and grown as described in; Evans et.al., J. Biol. Chem. 267: 10743-10751. These cells can be plated in either 100 mm or in 6-well (35-mm) culture plates from Falcon Scientific (VWR, Missisauga, ON) and maintained in minimal essential medium containing 5% human lipoprotein-deficient serum (LPDS). The appropriate concentrations (ranging from 0, 0.5, 1, 5, 10, and 50 pg/ml) of xanthohumol solubilized in dimethyl sulfoxide (DMSO) are added to the dishes and incubated for 24 hours. Duplicate dishes of HepG2 cells will be used for each time point or concentration of compound. Apo B secretion and triglyceride synthesis catalyzed by diacylglycerol acyltransferase (DGAT), primary processes associated with the secretion of LDL can be measured. Modulation of apoB secretion from HepG2 cells via HMG-CoA reductase inhibition by xanthohumol will indicate a significant decrease in apoB.

Incorporation of carbon 14 labeled acetic acid or carbon 14 labeled oleic acid into cellular lipids will be performed from 0 h to 5 h or from 19 h to 24 h after the addition of xanthohumol at different concentrations. This protocol will provide information about the inhibition of HMG-CoA reductase over time and will determine if differences in apoB secretion are due to a difference in the metabolism or clearance of inhibitor from the hepatocyte, resulting in an attenuation of HMG-CoA reductase inhibiton at later time points.

From this assay system, it can be determined that oleate-induced stimulation of apo B secretion was significantly decreased. This can be determined by radiolabeling 3H-oleic acid and measuring its incorporation into triglycerides, because fatty acids are synthesized into triglycerides. The data is expected to indicate that xanthohumol inhibits DGAT activity resulting in decreased synthesis of triglycerides. In addition, it will be observed that carbon 14 labeled oleic acid incorporation into cholesteryl ester (CE) will be decreased by xanthohumol during the incubation.

### EXAMPLE 2

Inhibition of hepatic ACAT will also be demonstrated in HEPG2 cells as evidenced by incorporation of carbon 14 labeled oleic acid or carbon 14 labeled acetic acid into cellular lipids by incubating xanthohumol in the assay, and measuring the incorporation of oleic acid into cholesteryl ester (CE) or phospholipid. Results will show a significant reduction in incorporation of radiolabelled acetate or oleate into cholesteryl ester. In other words, xanthohumol decreased cholesterol esterification. This is an indication that xanthohumol is an ACAT inhibitor.

DGAT inhibition my also be involved in improved glucose metabolism, which has implications for the treatment of diabetes. Recent research indicates that there are two forms of DGAT, DGAT1 and DGAT2, or two distinct DGAT genes.

Glucose (carbohydrate) and insulin each have effects on DGAT, glucose preferentially enhances DGAT1 mRNA expression, and insulin specifically increases the level of DGAT2 mRNA. Therefore, glucose and insulin help regulate the DGAT enzyme.

The prenylcalcones and prenylflavones have potential in the treatment of elevated cholesterol and other dyslipidaemias. By helping to control or lower cholesterol, the esterification of cholesterol, and triglyceride metabolism, as well as glucose, and insulin resistance, these compounds could be effectively used as broad spectrum cardiovascular agents.

It is anticipated that the HMG CoA reductase inhibitor from hops should inhibit the enzyme by at least 10%, and preferably by 25-75%. Complete inhibition of the enzyme may not be desirable due to potential unknown side-effects. By comparison, Atorvastatin inhibits HMG CoA reductase in HepG2 cells by about 96%. ACAT inhibition by xanthohumol in HepG2 cells is expected to be from 10-75%.

The dose of the prenylchalcone of flavonone is expected to be at least 5 to 1,000 mg. The dose of pure xanthohumol is expected to be lower than an extract of hops containing 3-5% xanthohumol. If an extract of hops is used, the dose would be 25-3,000 mg due to the low amount of xanthohumol. If purified xanthohumol is used, the dose may be from 5-1,000 mg, but more preferably about 5-500 mg.

Preferably, a dose of prenylchalcone such as xanthohumol would achieve a blood level of from at least 0.01 to 0.5 μg/ml. Or a blood level concentration of at least 10 to 200 μM.

The preferred embodiments may also employ conjugates of prenylchalcones or flavonones, or conjugates of xanthohumol.

Conjugates as used herein may mean prenylchalcones such as xanthohumol covalently bound or conjugated to a member selected from the group consisting of amino acids, sulfates, succinate, acetate, mono- or di-saccharides, or glutathione. A preferred conjugate would be a succinate such as xanthohumol succinate.

High concentrations of prenylchalcones or flavonones are expected to be contained in solvent based extracts of hops that result in high viscosity fluids (resin type materials) which can be further purified. This high viscosity extract can be combined with a pharmaceutically acceptable oil such as olive oil or soy phospholipids (phosphatidylcholine) and encapsulated in a soft gel capsule, or placed on a suitable pharmaceutical carrier to make a dry powder. If incorporated into a phospholipid complex, methods such as are described in U.S. Pat. Nos. 4,764,508; 4,963,527; and 5,043,323 may be used. Suitable carriers are maltodextrin, silica or salts of silica, talc, metal stearates, fibers such as guar gum, cellulose, modified cellulose (cellulose ethers), pectin, acacia, xanthum gum, or proteinaceous materials such as sodium casseinate, or casein, diatomacious earth, fullers earth, and gelatin. Beadlets of gelatin can be formed by heating a cooling the extract with gelatin to form beadlets using methods known to those skilled in the art. These carriers can be used individually or together in any number of combinations.

Pharmaceutical dosage forms such as capsules, tablets, or suppositories, can be made. Various excipients, such as cellulose or cellulose ethers, may be use to produce sustained-release of the active compound. The prenylchalcones and flavonones such as xanthohumol may also be formulated into a food, liquid drink, lozenge, gum or snack item.

According to the preferred embodiments, the animal may be selected form the group consisting of humans, non-humans primates, dogs, cats birds, horses or other warm blooded animals.

While the present invention is described above in connection with the preferred or illustrative embodiments, those embodiments are not intended to be exhaustive or limiting of the invention, but rather, the invention is intended to cover any alternatives, modifications or equivalents that may be included within its scope as defined by the appended claims.

What is claimed is:

1-24. (canceled)

25. A method of treating elevated blood cholesterol or dyslipidaemia, comprising administering to an animal an effective amount of a hops extract.

26. The method of claim 25, wherein the hops extract includes prenylated chalcones or prenylated flavones.

27. The method of claim 26, wherein the prenylated chalcones or prenylated flavones are selected from the group consisting of xanthohumol, xanthogalenol, desmethylxanthohumol (2′,4′,6′,4-tetrahydroxy-3-C-prenylchalcone), 2′,4′,6′,4-tetrahydroxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and combinations thereof.

28. The method of claim 26, wherein the prenylated chalcone or prenylated flavones is xanthohumol.

29. The method of claim 28, wherein the xanthohumol is administered in a single dose of from 5 mg to 1000 mg.

30. The method of claim 28, wherein the xanthohumol is administered in a single dose of from 5 mg to 750 mg.

31. The method of claim 28, wherein the xanthohumol is administered in a single dose of from 5 mg to 500 mg.

32. The method of claim 28, wherein the administration of xanthohumol provides a blood level of 0.01 to 0.5 μg/ml in the animal.

33. The method of claim 28, wherein the administration of xanthohumol provides a blood level concentration of 10 to 200 μM in the animal.

34. The method of claim 26, wherein the prenylated chalcones or prenylated flavones include a conjugate of xanthohumol.

35. The method of claim 34, wherein the conjugate of xanthohumol is xanthohumol succinate.

36. The method of claim 25, wherein the hops extract is administered in dosage forms selected from the group consisting of capsules, tablets, and suppositories.

37. The method of claim 25, wherein the hops extract is administered with an acceptable pharmaceutical carrier.

38. The method of claim 25, wherein the animal is a human.

39. A method of treating hyperlipidaemia, comprising administering to an animal an effective amount of a hops extract.

40. The method of claim 39, wherein the hops extract includes prenylated chalcones or prenylated flavones.

41. The method of claim 40, wherein the prenylated chalcones or prenylated flavones are selected from the group consisting of xanthohumol, xanthogalenol, desmethylxanthohumol (2′,4′,6′,4-tetrahydroxy-3-C-prenylchalcone), 2′,4′,6′,4-tetrahydroxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and combinations thereof.

42. The method of claim 41, wherein the prenylated chalcone or prenylated flavones is xanthohumol.

43. The method of claim 42, wherein the xanthohumol is administered in a single dose of from 5 mg to 1000 mg.

44. The method of claim 42, wherein the xanthohumol is administered in a single dose of from 5 mg to 750 mg.

45. The method of claim 42, wherein the xanthohumol is administered in a single dose of from 5 mg to 500 mg.

46. The method of claim 42, wherein the administration of xanthohumol provides a blood level of 0.01 to 0.5 μg/ml in the animal.

47. The method of claim 42, wherein the administration of xanthohumol provides a blood level concentration of 10 to 200 μM in the animal.

48. The method of claim 40, wherein the prenylated chalcones or prenylated flavones includes a conjugate of xanthohumol.

49. The method of claim 48, wherein the conjugate of xanthohumol is xanthohumol succinate.

50. The method of claim 39, wherein the hops extract is administered in dosage forms selected from the group consisting of capsules, tablets, and suppositories.

51. The method of claim 39, wherein the hops extract is administered with an acceptable pharmaceutical carrier.

52. The method of claim 39, wherein the animal is a human.
